(19) United States
(12) Patent Application Publication
(10) Pub. No.: US 2017/0165072 A1
(43) Pub. Date: Jun. 15, 2017

(54) BONE IMPLANT

(71) Applicant: Metal Industries Research & Development Centre, KAOHISUNG (TW)

(72) Inventors: Yao-Te PENG, KAOHISUNG (TW); Yau-Chia LIU, KAOHISUNG (TW); Tzyy-Ker SUE, KAOHISUNG (TW); Ho-Chung FU, KAOHISUNG (TW); Yen-Nien CHEN, KAOHISUNG (TW)

(21) Appl. No.: 14/991,601

(22) Filed: Jan. 8, 2016

(30) Foreign Application Priority Data
Dec. 9, 2015 (TW) 104141379

(51) Int. Cl. A61F 2/28; A61F 2/32

(52) U.S. Cl. CPC A61F 2/2803; A61F 2/2875; A61F 2/32; A61F 2220/0008; A61F 2220/0025

(57) ABSTRACT

A bone implant to be disposed between a first bone section and a second bone section is disclosed. The bone implant includes a body structure, a plurality of through-hole structures and an extended channel structure. The body structure has a first end surface to be connected to the first bone section and a second end surface to be connected to the second bone section. The plurality of through-hole structures are disposed in the body structure, pass through the first end surface and the second end surface and have a through-hole axis. The extended channel structure is disposed in the body structure, wherein the extended channel structure enables a new bone section to grow along a direction other than the through-hole axis between the first bone section and the second bone section.

### CROSS-REFERENCE TO RELATED APPLICATION AND CLAIM OF PRIORITY

The application claims the benefit of Taiwan Patent Application No. 104141379, filed on Dec. 9, 2015, at the Taiwan Intellectual Property Office, the disclosure of which is incorporated herein in its entirety by reference.

### FIELD OF THE INVENTION

The present invention relates to a bone implant, and more particularly to a bone implant with growth guidance.

### BACKGROUND OF THE INVENTION

The appearance of any bone implant is considered in bone repair. In addition, the duration of the bone implant is valued more and more. Currently, manufacturing bone implants requires reconstructing the appearance of the bone via a CT image, manufacturing the model of the bone implant via computer files in advance, and casting the bone cement in the model to obtain a bone implant applicable for bone repair (TW I469767 and TW I324509). Although this method can achieve the purpose of repairing the appearance quickly, the repair strength and the function of the bone implant still can not satisfy some requirements of clinical use. Due to current progress in the process technology, the most common structure of bone implant is a multi-hole titanium mesh sheet bent to repair and fix the broken bone (U.S. Ser. No. 10/517,843 and U.S. Ser. No. 11/289,591). Compared with bone cement, this structure provides stronger supporting strength. However, the process of bending may result in a limitation to the fixation, and the multi-hole titanium mesh sheet can not provide enough fixation and allow the patient to wear a denture due to the large area to be repaired in a mandible (TW I496557). Implants of imitation bone are used in spine treatment, the method thereof usually uses a regular interbody fusion cage, the hollow shape thereof is used to fix the bone, and the hollow shape design can reduce the problem of stress concentration (TW M273326). If the holes in the interbody fusion cage are too large, the effect of osseointegration will be worse, and the process of manufacturing the interbody fusion cage is complex. In addition, the multi-hole implant for supporting the spine (TW 201521670) is one implant design disposed with different sized holes inside and outside. Although this design can achieve the purpose of obtaining a similar bone structure and reducing stress while supporting the spine, this design can not provide holes for fixation, and can only be fixed via additional fixing devices, and the shape thereof can not conform to the anatomical shape of the bone.

In order to overcome the drawbacks in the prior art, a bone implant is disclosed. The particular design in the present invention not only solves the problems described above, but is also easy to implement. Thus, the present invention has utility for industry.

### SUMMARY OF THE INVENTION

In accordance with one aspect of the present invention, a bone implant to be disposed between a first bone section and a second bone section is disclosed. The bone implant includes a body structure, a plurality of through-hole structures and an extended channel structure. The body structure has a first end surface to be connected to the first bone section and a second end surface to be connected to the second bone section. The plurality of through-hole structures are disposed in the body structure, pass through the first end surface and the second end surface and have a through-hole axis. The extended channel structure is disposed in the body structure, wherein the extended channel structure enables a new bone section to grow along a direction other than the through-hole axis between the first bone section and the second bone section.

In accordance with another aspect of the present invention, a bone implant for implantation onto a to-be-repaired bone section is disclosed, wherein the to-be-repaired bone section has an anatomical direction. The bone implant includes a body structure, a plurality of through-hole structures and an extended channel structure. The body structure has an end surface for connecting the to-be-repaired bone section. The plurality of through-hole structures are disposed in the body structure and have an anatomical direction axis conforming to the anatomical direction. The extended channel structure is disposed in the body structure, and enables a new bone section to grow along a first direction other than that of the anatomical direction axis.

In accordance with a further aspect of the present invention, a bone implant for connecting to a to-be-repaired bone section is disclosed, wherein the to-be-repaired bone section has an anatomical direction. The bone implant includes a body structure connected to the to-be-repaired bone section, and a channel structure disposed in the body structure, wherein the channel structure is one of a two-dimensional and three-dimensional grid structures, and enables a new bone to grow to fill therein.

The objectives and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 shows a stereogram of a bone implant with growth guidance according to a preferred embodiment of the present invention;

FIG. 2 shows a diagram of a bone implant disposed in the mandible;

FIG. 3 shows a vertical sectional view of a bone implant according to a preferred embodiment of the present invention;

FIG. 4 shows a vertical sectional view of a bone implant according to another preferred embodiment of the present invention;

FIG. 5 shows a diagram of a three-dimensional grid structure;

FIG. 6 shows a diagram of a bone implant disposed in the facial bone; and

FIG. 7 shows a diagram of a bone implant disposed in the hip bone.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; they are not intended to be exhaustive or to be limited to the precise form disclosed.

The present invention has the following advantages: (1) a design that guides bone growth and enables bone cells to grow into the channels conforming to the anatomical direction to enhance the stability of the repaired bone; (2) the holes disposed on the surface of the bone implant can be applied to wearing a denture so that the bite function can be restored immediately after repairing the broken mandible, and the holes can also implement the fixation right away; (3) the hole structure design in the bone implant can reduce stress concentration and increase the space for bone growth.

### Embodiment 1

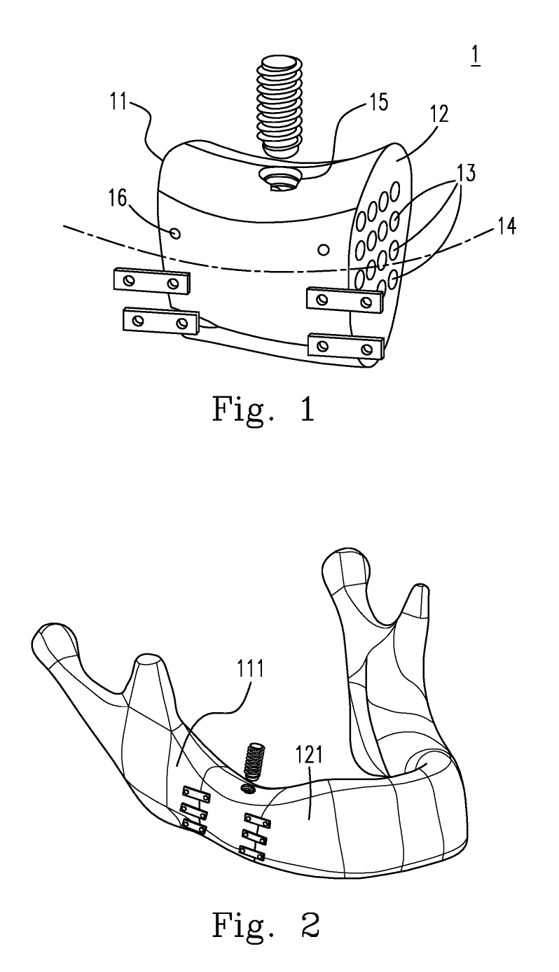

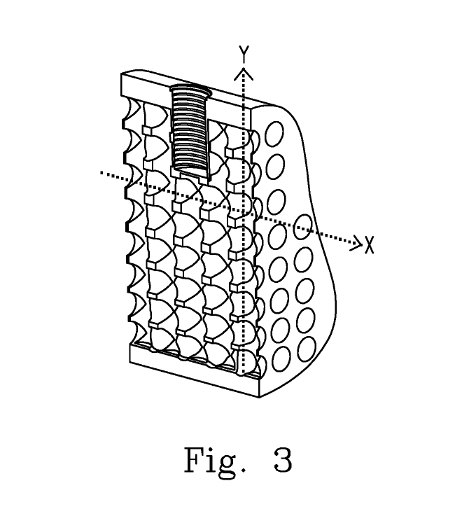

Please refer to FIG. 1, which shows the stereogram of a bone implant 1 with growth guidance according to a preferred embodiment of the present invention. Please refer to FIG. 2. The bone implant 1 is disposed between a first bone section 111 and a second bone section 121. The bone implant 1 has a first end surface 11 and a second end surface 12, wherein the first end surface 11 is connected to the first bone section 111 and the second end surface 12 is connected to the second bone section 121. The bone implant 1 further has a plurality of through-hole structures 13 and a through-hole axis 14, wherein the direction of the through-hole axis 14 is along the anatomical direction of the bone shape, and the plurality of through-hole structures 13 pass through the first end surface 11 and the second end surface 12 along the through-hole axis 14. Please refer to FIG. 3, which shows the vertical sectional view of the bone implant 1. As shown in FIG. 3, there are not only the plurality of through-hole structures 13 in the bone implant 1, but also an extended channel structure disposed therein extending along the Y direction so that the bone cells from the first bone section 111 and the second bone section 121 can grow into the bone implant 1 along the plurality of through-hole structures 13, and the growth can be further guided via the extended channel structure. The extended channel structure is a channel structure in the bone implant 1, and intersects with the plurality of through-hole structures 13 at fixed intervals, wherein the intersectional angle can be less than 90°, equal to 90° or more than 90°. Each of the plurality of through-hole structures 13 and the extended channel structure has a hole size ranging between 200 μm and 750 μm, and the shape of the hole is circular, rectangular or irregular. Please refer to FIG. 1 again. There are different sized holes distributed on the surface of the bone implant 1, wherein the inner wall of the holes has threads disposed thereon. The larger hole 15 can mark the corresponding denture position by using the patient's mouth bite information so that the patient can wear a denture immediately after the surgical operation on the mandible. The smaller holes 16 are distributed in several locations on the bone implant 1 to connect to the broken bone by using commercial bone screws, bony plates or the combination thereof to enhance stability in the early days of recovery. The material of the bone implant 1 is titanium, titanium alloy, stainless steel or ceramic.

### Embodiment 2

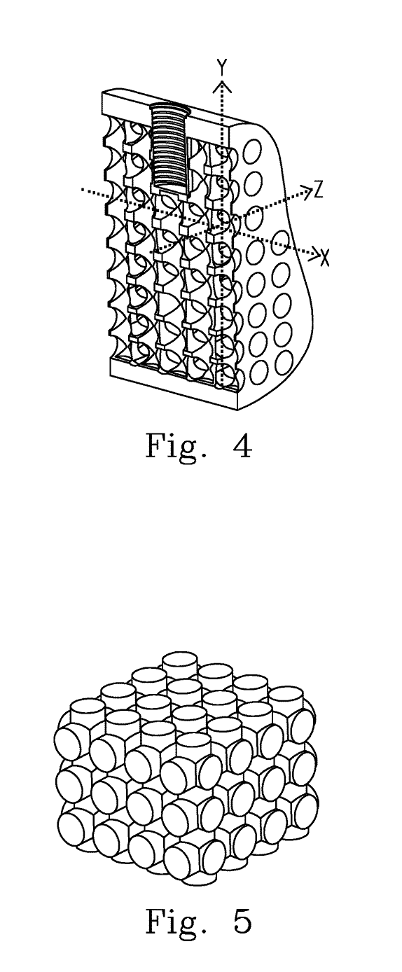

Please refer to FIG. 1, which shows the stereogram of a bone implant 1 with growth guidance according to a preferred embodiment of the present invention. Please refer to FIG. 2. The bone implant 1 is disposed between a first bone section 111 and a second bone section 121. The bone implant 1 has a first end surface 11 and a second end surface 12, wherein the first end surface 11 is connected to the first bone section 111 and the second end surface 12 is connected to the second bone section 121. The bone implant 1 further has a plurality of through-hole structures 13 and a through-hole axis 14, wherein the direction of the through-hole axis 14 is along the anatomical direction of the bone shape, and the plurality of through-hole structures 13 pass through the first end surface 11 and the second end surface 12 along the through-hole axis 14. Please refer to FIG. 4, which shows the vertical sectional view of the bone implant 1. As shown in FIG. 4, there are not only the plurality of through-hole structures 13 in the bone implant 1, but also an extended channel structure disposed therein extending along the Y and Z directions so that the bone cells from the first bone section 111 and the second bone section 121 can grow into the bone implant 1 along the plurality of through-hole structures 13, and the growth can be further guided via the extended channel structure. The extended channel structure is a channel structure in the bone implant 1, and intersects with the plurality of through-hole structures 13 at a fixed interval to construct a 3-D hole observable in the directions of up and down, front and rear, and left and right to form a three-dimensional grid structure whose diagram is shown in FIG. 5. The angle between any two dimensions of the three-dimensional grid structure can be less than 90°, equal to 90° or more than 90°. Each of the plurality of through-hole structures 13 and the extended channel structure has a hole size ranging between 200 μm and 750 μm, and the shape of the hole is circular, rectangular or irregular. Please refer to FIG. 1 again. There are different sized holes distributed on the surface of the bone implant 1, wherein the inner wall of the holes has threads disposed thereon. The larger hole 15 can mark the corresponding denture position by using the patient's mouth bite information so that the patient can wear a denture immediately after the surgical operation on the mandible. The smaller holes 16 are distributed in several locations on the bone implant 1 to connect to the broken bone by using commercial bone screws, bony plates or the combination thereof to enhance stability in the early days of recovery. The material of the bone implant 1 is titanium, titanium alloy, stainless steel or ceramic.

### Embodiment 3

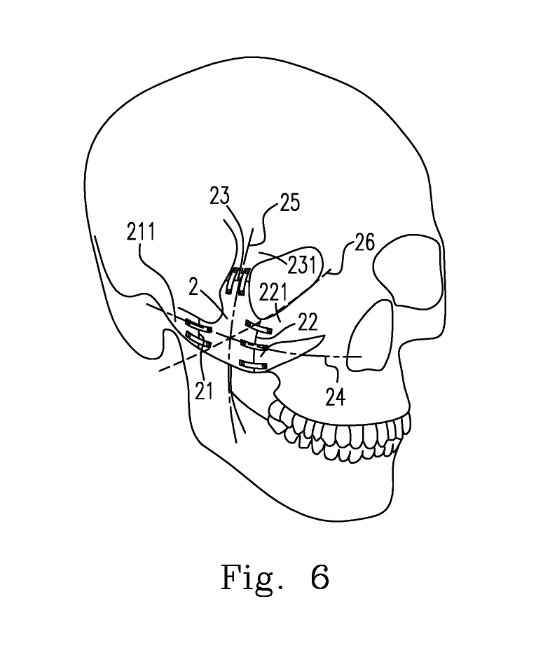

Please refer to FIG. 6, which shows the diagram of a bone implant 2 with growth guidance disposed in the facial bone according to another preferred embodiment of the present invention. As shown in FIG. 2, the bone implant 2 is disposed among a first bone section 211, a second bone section 221 and a third bone section 231. The bone implant 2 has a first end surface 21, a second end surface 22 and a third end surface 23, wherein the first end surface 21 is connected to the first bone section 211, the second end surface 22 is connected to the second bone section 221, and the third end surface 23 is connected to the third bone section 231. All of the first end surface 21, the second end surface 22 and the third end surface 23 have a plurality of holes. The plurality of holes represent openings in the plurality of through-hole structures (not shown) on the first end surface 21, the second end surface 22 and the third end surface 23. The plurality of holes extends to form channels, i.e. the plurality of through-hole structures, in the bone implant 2 along the anatomical direction axes 24 and 25. There is also an extended channel structure (not shown) disposed therein extending along an extended channel axis 26 so that the bone cells from the first bone section 211, the second bone section 221 and the third bone section 231 can grow into the bone implant 2 along the plurality of through-hole structures, and the growth can be further guided via the extended channel structure. The extended channel structure is a channel structure in the bone implant 2, and intersects with the plurality of through-hole structures at fixed intervals to construct a 3-D hole observable in the directions of up and down, front and rear, and left and right to form a three-dimensional grid structure whose diagram is shown in FIG. 5. The angle between any two dimensions of the three-dimensional grid structure can be less than 90°, equal to 90° or more than 90°. Each of the plurality of through-hole structures and the extended channel structure has a hole size ranging between 200 μm and 750 μm, and the shape of the hole is circular, rectangular or irregular. There are a plurality of holes distributed in several locations on the bone implant 2 to connect to the broken bone by using commercial bone screws, bony plates or the combination thereof to enhance stability in the early days of recovery.

### Embodiment 4

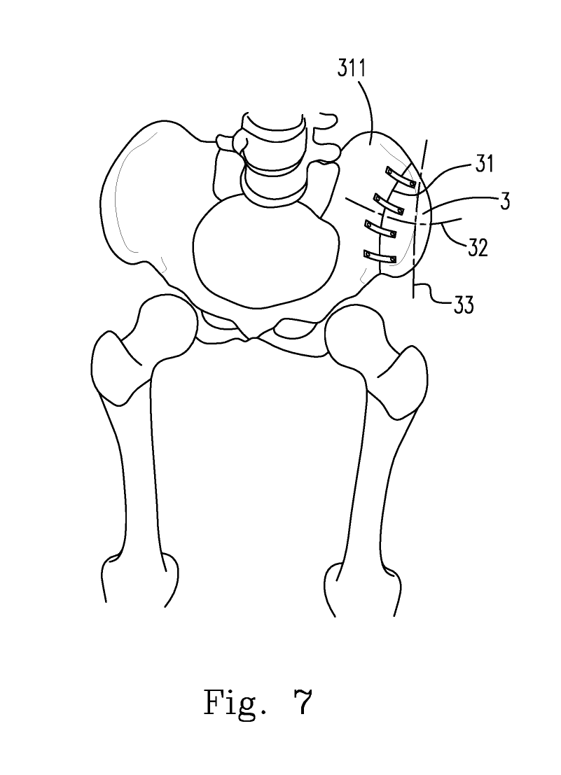

Please refer to FIG. 7, which shows the diagram of a bone implant 3 with growth guidance disposed in the hipbone according to a further preferred embodiment of the present invention. As shown in FIG. 7, the bone implant 3 is used to connect to a bone section 311. The bone implant 3 has an end surface 31, wherein the end surface 31 is connected to the bone section 311. The end surface 31 has a plurality of holes. The plurality of holes are openings in the plurality of through-hole structures (not shown) on the end surface 31. The plurality of holes extends to form channels, i.e. the plurality of through-hole structures, in the bone implant 3 along the anatomical direction axis 32. There is also an extended channel structure (not shown) disposed therein extending along an extended channel axis 33 so that the bone cells from the bone section 311 can grow into the bone implant 3 along the plurality of through-hole structures, and the growth can be further guided via the extended channel structure. The extended channel structure is a channel structure in the bone implant 3, and intersects with the plurality of through-hole structures at fixed intervals, wherein the intersectional angle can be less than 90°, equal to 90° or more than 90°. Each of the plurality of through-hole structures and the extended channel structure has a hole size ranging between 200 μm and 750 μm, and the shape of the hole is circular, rectangular or irregular. There are a plurality of holes distributed in several locations on the bone implant 3 to connect to the broken bone by using commercial bone screws, bony plates or the combination thereof to enhance stability in the early days of recovery.

### Embodiments

1. A bone implant to be disposed between a first bone section and a second bone section includes a body structure, a plurality of through-hole structures and an extended channel structure. The body structure has a first end surface to be connected to the first bone section and a second end surface to be connected to the second bone section. The plurality of through-hole structures is disposed in the body structure, passes through the first end surface and the second end surface and has a through-hole axis. The extended channel structure is disposed in the body structure, wherein the extended channel structure enables a new bone section to grow along a direction other than the through-hole axis between the first bone section and the second bone section.

2. In the bone implant according to Embodiment 1, each of the plurality of through-hole structures and the extended channel structure has a hole size ranging between 200 μm and 750 μm.

3. In the bone implant according to Embodiments 1-2, each of the plurality of through-hole structures and the extended channel structure has a shape selected from a group consisting of circular, rectangular and irregular shapes.

4. In the bone implant according to Embodiments 1-3, the plurality of through-hole structures intersects the extended channel structure at fixed intervals.

5. In the bone implant according to Embodiments 1-4, the plurality of through-hole structures and the extended channel structure construct a 3-D hole observable in the directions of up and down, front and rear, and left and right to form a three-dimensional grid structure.

6. In the bone implant according to Embodiments 1-5, any two dimensions of the three-dimensional grid structure has therebetween an angle being selected from a group consisting of less than 90°, equal to 90° and more than 90°.

7. In the bone implant according to Embodiments 1-6, the body structure has a surface disposed with holes having one of the same size and different sizes.

8. In the bone implant according to Embodiments 1-7, each of the holes has an inner wall having threads disposed thereon.

9. In the bone implant according to Embodiments 1-8, the holes are used to wear a denture.

10. In the bone implant according to Embodiments 1-9, the holes are used to fix the bone implant via one selected from a group consisting of a bone screw, a bony plate and the combination thereof.

11. In the bone implant according to Embodiments 1-10, the bone implant is made of a material being one selected from a group consisting of titanium, titanium alloy, stainless steel and ceramic.

12. A bone implant for implantation to a to-be-repaired bone section includes a body structure, a plurality of through-hole structures and an extended channel structure, wherein the to-be-repaired bone section has an anatomical direction. The body structure has an end surface for connecting the to-be-repaired bone section. The plurality of through-hole structures is disposed in the body structure and has an anatomical direction axis conforming to the anatomical direction. The extended channel structure is disposed in the body structure, and enables a new bone section to grow along a first direction other than that of the anatomical direction axis.

13. In the bone implant according to Embodiment 12, the bone implant further includes a second extended channel structure disposed in the body structure and enables a new bone section to grow along a second direction other than those of the anatomical direction axis and the first direction.

14. In the bone implant according to Embodiments 12-13, the extended channel structure has a first extended channel axis, the second extended channel structure has a second extended channel axis, and any two of the anatomical direction axis, the first extended channel axis and the second extended channel axis are not parallel to each other.

15. In the bone implant according to Embodiments 12-14, the new bone section grows in the plurality of through-hole structures, the extended channel structure and the second extended channel structure.

16. A bone implant for connecting to a to-be-repaired bone section includes a body structure and a channel structure, wherein the to-be-repaired bone section has an anatomical direction. The body structure is connected to the to-be-repaired bone section. The channel structure is disposed in the body structure, is one of a two-dimensional and three-dimensional grid structures, and enables a new bone to grow to fill therein.

17. In the bone implant according to Embodiment 16, the channel structure has a plurality of channels, and only a portion of the plurality of channels extends along the anatomical direction.

18. In the bone implant according to Embodiments 16-17, each of the plurality of channels has a section diameter ranging between 200 μm and 750 μm.

19. In the bone implant according to Embodiments 16-18, any two channels intersect to form an angle being one selected from a group consisting of less than 90°, equal to 90° and more than 90°.

20. In the bone implant according to Embodiments 16-19, the body structure has a surface disposed with a plurality of holes to fix the bone implant via one selected from a group consisting of a bone screw, a bony plate and the combination thereof.

Based on the above, the present invention effectively solves the problems and drawbacks in the prior art, and thus it meets the demands of the industry and is industrially valuable.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A bone implant to be disposed between a first bone section and a second bone section, comprising:
a body structure having a first end surface to be connected to the first bone section and a second end surface to be connected to the second bone section;
a plurality of through-hole structures disposed in the body structure, passing through the first end surface and the second end surface and having a through-hole axis; and
an extended channel structure disposed in the body structure, wherein the extended channel structure enables a new bone section to grow along a direction other than the through-hole axis between the first bone section and the second bone section.

2. The bone implant as claimed in claim 1, wherein each of the plurality of through-hole structures and the extended channel structure has a hole size ranging between 200 μm and 750 μm.

3. The bone implant as claimed in claim 2, wherein each of the plurality of through-hole structures and the extended channel structure has a shape selected from a group consisting of circular, rectangular and irregular ones.

4. The bone implant as claimed in claim 1, wherein the plurality of through-hole structures intersect the extended channel structure at a fixed interval.

5. The bone implant as claimed in claim 4, wherein the plurality of through-hole structures and the extended channel structure construct a 3-D hole observable in the directions of up and down, front and rear, and left and right to form a three-dimensional grid structure.

6. The bone implant as claimed in claim 5, wherein any two dimensions of the three-dimensional grid structure have therebetween an angle being selected from a group consisting of less than 90°, equal to 90° and more than 90°.

7. The bone implant as claimed in claim 1, wherein the body structure has a surface disposed with holes having one of the same size and different sizes.

8. The bone implant as claimed in claim 7, wherein each of the holes has an inner wall having threads disposed thereon.

9. The bone implant as claimed in claim 8, wherein the holes are used to wear a denture.

10. The bone implant as claimed in claim 8, wherein the holes are used to fix the bone implant via one selected from a group consisting of a bone screw, a bony plate and the combination thereof.

11. The bone implant as claimed in claim 1, wherein the bone implant has a material being one selected from a group consisting of titanium, titanium alloy, stainless steel and ceramic.

12. A bone implant for implantation to a to-be-repaired bone section, wherein the to-be-repaired bone section has an anatomical direction, comprising:
a body structure having an end surface for connecting the to-be-repaired bone section;
a plurality of through-hole structures disposed in the body structure and having an anatomical direction axis conforming to the anatomical direction; and
an extended channel structure disposed in the body structure, and enabling a new bone section to grow along a first direction other than that of the anatomical direction axis.

13. The bone implant as claimed in claim 12, wherein the bone implant further comprises a second extended channel structure disposed in the body structure and enabling a new bone section to grow along a second direction other than those of the anatomical direction axis and the first direction.

14. The bone implant as claimed in claim 13, wherein the extended channel structure has a first extended channel axis, the second extended channel structure has a second extended channel axis, and any two of the anatomical direction axis, the first extended channel axis and the second extended channel axis are not parallel to each other.

15. The bone implant as claimed in claim 13, wherein the new bone section grows in the plurality of through-hole structures, the extended channel structure and the second extended channel structure.

16. A bone implant for connecting to a to-be-repaired bone section, wherein the to-be-repaired bone section has an anatomical direction, comprising:
a body structure connected to the to-be-repaired bone section;
a channel structure disposed in the body structure, being one of a two-dimensional and three-dimensional grid structures, and enabling a new bone to grow to fill therein.

17. The bone implant as claimed in claim 16, wherein the channel structure has a plurality of channels, and only a portion of the plurality of channels extends along the anatomical direction.

18. The bone implant as claimed in claim 17, wherein each of the plurality of channels has a section diameter ranging between 200 μm and 750 μm.

19. The bone implant as claimed in claim 17, wherein any two channels intersect to form an angle being one selected from a group consisting of less than 90°, equal to 90° and more than 90°.

20. The bone implant as claimed in claim 16, wherein the body structure has a surface disposed with a plurality of holes to fix the bone implant via one selected from a group consisting of a bone screw, a bony plate and the combination thereof.
